(19) United States
(12) Patent Application Publication
(10) Pub. No.: US 2010/0213081 A1
(43) Pub. Date: Aug. 26, 2010

(54) APPARATUS AND METHOD FOR DETECTING PRESENCE OR DETERMINING BIOLOGICALLY SPECIFICALLY REACTIVE SUBSTANCE

(75) Inventors: Nobuhiko Ozaki, Kanagawa (JP); Shinki Kojima, Kanagawa (JP); Tetsuo Yukimasa, Kanagawa (JP)

Correspondence Address: GREENBLUM & BERNSTEIN, P.L.C., 1950 ROLAND CLARKE PLACE, RESTON, VA, 20191, US

(73) Assignee: PANASONIC CORPORATION, Osaka (JP)

(21) Appl. No.: 12/679,339

(22) PCT Filed: Oct. 10, 2008
(86) PCT No.: PCT/JP2008/002884

§ 371 Date: Mar. 22, 2010

(30) Foreign Application Priority Data
Oct. 22, 2007 (JP) 2007-274194

(51) Int. Cl. G01N 27/26; G01N 33/487

(52) U.S. Cl. 205/777.5; 204/403.01; 205/792

(57) ABSTRACT

This invention provides an apparatus for determining a biologically specifically reactive substance using the acceleration of a biologically specific reaction by pearl chaining of carrier particles. The apparatus can suppress the generation of heat caused by the pearl chaining to suppress the vaporization of a sample solution. The apparatus comprises a substrate comprising a pair of electrodes and a reaction field disposed between the pair of electrodes, voltage applying means for applying an alternating voltage to the pair of electrodes, and carrier particles which are disposed in the reaction field and undergo pearl chaining upon exposure of an alternating electric field applied to the reaction field by the alternating voltage. The apparatus is characterized in that the voltage applying means applies two or more alternating voltages different from each other in amplitude.

### TECHNICAL FIELD

The present invention relates to a measuring apparatus and a measuring method using a test chip that detects a reaction product generated in a biological reaction, such as an immune reaction (antigen-antibody reaction) and a gene reaction.

### BACKGROUND ART

In recent years, various diagnostic chips have been developed. Most of these check chips are card devices called “μ-TAS” (Micro Total Analysis System), which have miniature flow-path structure.

A miniaturized flow path is very useful because the required amount of a sample extracted from a living organism is small. Also, when an entire apparatus including a health check chip can be made small by miniaturizing the flow path, the apparatus can be used in POCT (Point of Care Test) that allows diagnosis in doctor's offices and households, not only in relatively large-scale hospitals.

In medical fields or biochemical fields, an antigen or antibody is examined by detecting an agglutination substance generated by an antigen-antibody reaction, and a disease is diagnosed or analyzed (see Patent Documents 1 to 3). According to a method disclosed in Patent Document 1, an alternating voltage is applied to a reaction solution containing a carrier (e.g. styrene-based polymer particle), which supports a reactive substance such as an antigen and an antibody, and carrier particles are continuously aligned in a direction parallel to an electric field (which is also called “pearl chaining”). In addition, an antigen-antibody reaction is quantitated with agglutination degree of carrier particles after the voltage application is stopped. One characteristic of the alternating voltage applied in this measurement is that positive and negative amplitude values are periodically alternate and continuous. By this alternating voltage signal, carrier particles can be formed into pearl chains even in the presence of a salt, without being electrolyzed, in comparison with a DC pulse signal of the related art.

However, it has been difficult to accelerate pearl chaining of carrier particles. To accelerate pearl chaining of carrier particles, a higher dielectrophoretic force may be applied to carrier particles, and an alternating electric field of a reaction field may be stronger. A method for increasing the intensity of the alternating electric field is required either 1) to increase the amplitude of an applied alternating voltage or 2) to reduce a reaction field to which a voltage is applied. However, if one of them is carried out, more heat is generated in the reaction system. That is, energy of the alternating voltage is converted into heat energy, and the heat energy increases in proportion to conductivity of a solution. When the applied alternating voltage increases, the heat energy generated in the reaction system increases, and temperature increases. On the other hand, if the reaction field to which the voltage is applied is reduced, conductivity of the entire reaction system increases. As a result, heat energy generated in the reaction system increases, and temperature increases. In particular, this trend is distinct in a solution with high conductivity, and the solution is instantaneously vaporized in the reaction system. Thus, in many cases, pearl chaining of particles could not be used to measure components of a sample solution.

To form pearl chains of carrier particles, there is a limitation in materials forming the reaction field. A measuring apparatus forming a reaction field disclosed in Patent Document 1 is shown in FIG. 1. Slide glasses 101 and 102 form top and bottom of the reaction field, and electrodes 103 and 104 made of a conductor form sides of the reaction field. A volume of the reaction field is defined by setting a thickness of the electrodes to 0.02 mm and a distance between the electrodes to 0.5 mm. A member forming the reaction field is a glass or a conductive electrode, and the glass or the conductive electrode must be made of a material having high heat conductivity. However, when those materials are used, problems often occur in productivity. On the other hand, if the reaction field is formed of a resin material, it is inexpensive and easy to produce. However, the heat conductivity of the resin material is low, and therefore the temperature of the reaction system is remarkably increased in the reaction field where pearl chains are formed.

### Patent Document 1: Japanese Patent Application Laid-Open No. 7-83928

### Patent Document 2: Japanese Patent Application Laid-Open No. 2003-75441

### Patent Document 3: Japanese Patent Application Laid-Open No. 59-173761

### DISCLOSURE OF INVENTION

### Problems to be Solved by the Invention

Further, when the above-described measuring method using the pearl chaining of carrier particles is applied to measure components of a blood sample or a blood plasma sample, which are examples of a biological sample, the components cannot be appropriately measured in some cases. The blood sample or the blood plasma sample is a solution that generally has substantially the same salt concentration (about 120 mM) as a normal saline solution and has high conductivity. For this reason, heat generated in the reaction system by pearl chaining denatures proteins contained in the sample and the denatured proteins are fixed in the reaction system, or the proteins themselves, which are subjected to measurement, are denatured, to result in having a harmful effect of not exhibiting a specific reaction such as an antigen-antibody reaction. To avoid these, an additional process such as diluting the blood plasma sample with a salt-free solution in advance, is required.

It is therefore an object of the present invention to provide an apparatus and method that can suppress the generation of heat by forming pearl chains and suppress the vaporization of a sample solution, in the apparatus and method for detecting or measuring a biologically specifically reactive substance using acceleration of a biologically specific reaction by forming pearl chains of carrier particles. In addition, it is another object of the present invention to provide an apparatus and method in which a high-conductivity solution represented by a blood solution or a blood plasma solution is used as a measuring solution.

### Means for Solving the Problems

The first aspect of the present invention relates to the following apparatus.

[1] An apparatus for detecting or measuring a biologically specifically reactive substance by a biologically specific agglutination reaction on carrier particles, the apparatus including: a device including a pair of electrodes and a reaction field disposed between the pair of electrodes; and a voltage applying section that applies an alternating voltage to the pair of electrodes to form pearl chains of the carrier particles disposed in the reaction field, wherein the voltage applying section applies at least two alternating voltages having different amplitudes.
[2] The apparatus described in [1], wherein the voltage applying section alternately applies a first alternating voltage with a first amplitude, and a second alternating voltage with a second amplitude, and the first alternating voltage applies a first alternating electric field having an electric field intensity of 20 V/mm or more and 100 V/mm or less is applied to the reaction field, and the second alternating voltage applies a second alternating electric field having an electric field intensity lower than the first electric field intensity is applied to the reaction field.
[3] The apparatus described in [2], wherein the amplitude of the second alternating voltage is 0 V.
[4] The apparatus described in [2] or [3], wherein a repetition period including a time period the first alternating voltage is applied and a time period the second alternating voltage is applied is 30 nanoseconds or more and 5 seconds or less.
[5] The apparatus described in [4], wherein the ratio between the time period the first alternating voltage is applied and the repetition period is 75% to 95%.
[6] The apparatus described in any one of [1] to [5], wherein the device includes a flow path forming the reaction field, and at least one surface of the flow path is formed of a resin.
[7] The apparatus described in [6], wherein the device includes: a top substrate forming a top surface of the flow path, a substrate shape of a top surface side being a flat panel; a bottom substrate forming a bottom surface of the flow path, a substrate shape of the bottom surface side being a flat panel; an middle substrate forming a side surface of the flow path and including a penetrating region corresponding to the shape of the flow path, and wherein at least the middle substrate is formed of a resin.
[8] The apparatus described in [7], wherein a surface of the middle substrate which does not form the flow path is adhesive.
[9] The apparatus described in [6], wherein all surfaces of the flow path is formed of a resin.

A second aspect of the present invention relates to a method described below.

[10] A method for detecting or measuring presence of a biologically specifically reactive substance, the method including steps of: forming pearl chains of carrier particles by applying an alternating voltage to a reaction system, which includes the carrier particles and a target solution containing the biologically specifically reactive substance capable of a biologically specific agglutination reaction on the carrier particles, wherein the reaction system receives at least two alternating electric fields having different electric field intensities.
[11] The method described in [10], wherein when the alternating electric fields are applied, an effective value of a current flowing in the target solution may be 0.7 mA or more and 96 mA or less.
[12] The method described in [10] or [11], wherein the target solution has a conductivity of 0.1 mS/cm or more and 35 mS/cm or less.
[13] The method described in any one of [10] to [12], wherein when the alternating electric fields are applied, a temperature of the target solution is maintained at 45° C. or less.
[14] The method described in any one of [10] to [13], wherein the target solution is blood or blood plasma.

### ADVANTAGEOUS EFFECTS OF THE INVENTION

In the apparatus according to the present invention, the voltage applying section applies two or more alternating electric fields including a first alternating electric field and a second alternating electric field, to the reaction field. The first alternating electric field provides a dielectrophoretic force to carrier particles for alignment (pearl chaining) in a pearl chain shape. Meanwhile, the second alternating electric field has an electric field intensity lower than the first alternating electric field, so that it is possible to release heat generated in the reaction system by forming pearl chains to the outside.

For this reason, generation of heat by forming pearl chains of the carrier particles is suppressed, and the solution is not vaporized even in a case of a high-conductivity sample solution, and furthermore, the carrier particles may be rapidly formed into pearl chains. Consequently, according to the present invention, it is possible to measure components of the blood or blood plasma, which is very conductive, and which is an example of a biological sample, without diluting blood or blood plasma in advance. Therefore, it is possible to provide a measuring apparatus in a simple structure, without providing a previous diluting means. Also, the operator is not requested to dilute the blood plasma in advance, so that it is possible to conduct measurement simply without errors.

As described above, by applying the second alternating electric field in addition to the first alternating electric field for causing the pearl chaining, generation of heat in the reaction system is suppressed. Therefore, the apparatus in which the reaction field is configured may be formed of a resin with much poorer heat conduction than glass, as well as glass with high conduction. Accordingly, the apparatus may be produced at low cost, and the production is simplified, thereby remarkably improving production efficiency.

Moreover, by appropriately adjusting the electric field intensities and the application time period of the first alternating electric field and the second alternating electric field, the time to complete the pearl chaining of the carrier particles need not be extended greatly, so that it is possible to achieve rapid measurement.

### BRIEF DESCRIPTION OF DRAWINGS

FIG. 1 shows a configuration of a conventional substrate;

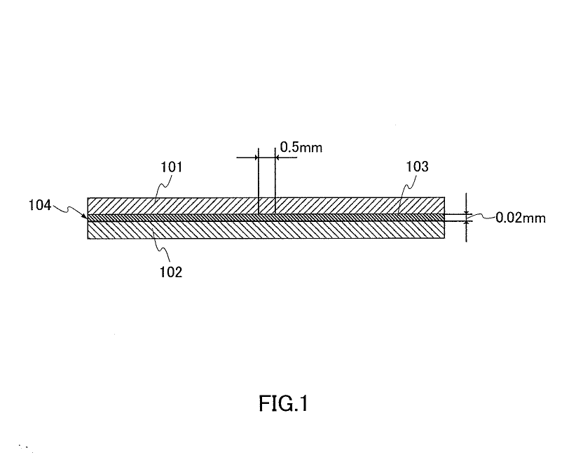

FIG. 2 is a configuration diagram of apparatus 1 according to the present invention;

FIG. 3 is a partial plan view of device 2 included in apparatus 1 according to the present invention;

FIG. 4A is a partial cross-sectional view taken along line A-A′ of FIG. 3, which shows an example of device 2 shown in FIG. 2;

FIG. 4B is a partial cross-sectional view taken along line A-A′ of FIG. 3, which shows another example of device 2 shown in FIG. 2;

FIG. 5A is a schematic diagram illustrating an example of an alternating voltage waveform of an alternating voltage applied by a voltage applying section of apparatus 1 according to the present invention;

FIG. 5B is a schematic diagram illustrating another example of an alternating voltage waveform of an alternating voltage applied by the voltage applying section of apparatus 1 according to the present invention;

FIG. 6 is a flow chart to explain an example of a method according to an embodiment of the present invention; and

FIG. 7 is a graph showing the result of Example 3.

### BEST MODE FOR CARRYING OUT THE INVENTION

### 1. Apparatus According to the Present Invention

A detecting or measuring apparatus of the present invention includes: 1) a device having a pair of electrodes and a reaction field disposed between the pair of electrodes; and 2) a voltage applying section configured to apply an alternating voltage to the pair of electrodes such that pearl chains of carrier particles disposed in the reaction field are formed.

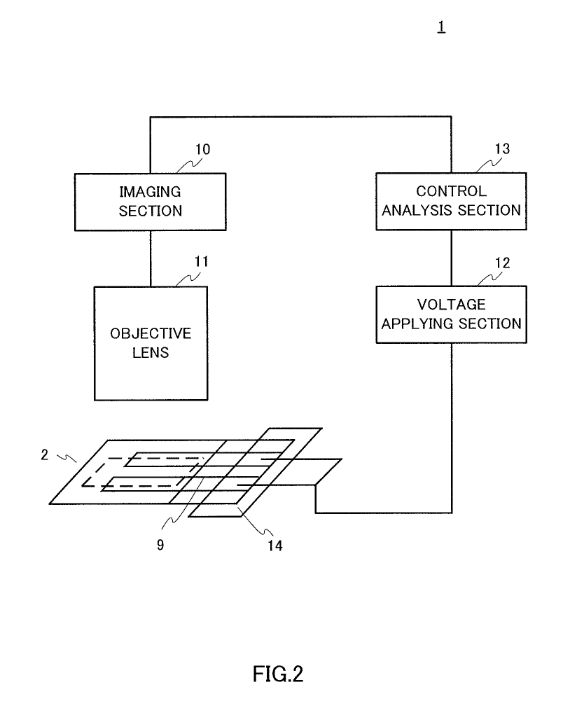

FIG. 2 shows a schematic structure of an example of the apparatus (apparatus 1) according to the present invention.

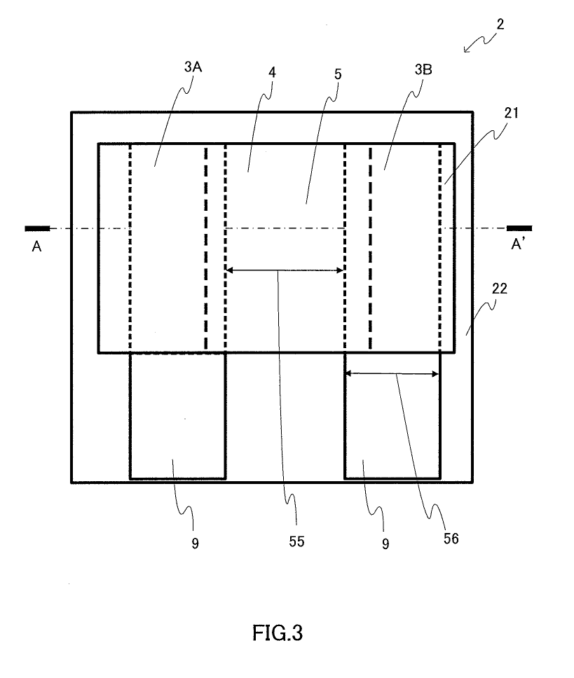

Apparatus 1 includes device 2 and voltage applying section 12. FIG. 3 is a plan view of device 2. Device 2 has a pair of electrodes 3A and 3B (i.e. electrode pair) connected to terminals 9. A reaction field is disposed between the electrode pair. The reaction field of device 2 is served as flow path 4 for disposition of liquid 5.

Voltage applying section 12 has connector 14. By connecting connector 14 of voltage applying section 12 to terminal 9 of device 2, a predetermined voltage can be applied between electrode 3A and electrode 3B. By an alternating voltage applied to the electrode pair, an alternating electric field is applied to the reaction field of device 2.

Distance 55 between electrode 3A and electrode 3B of device 2 is fixed. For this reason, voltages applied to electrode 3A and electrode 3B control the intensity of the alternating electric field applied to the reaction field. Therefore, it is possible to change the electric field intensity easily.

In addition, apparatus 1 shown in FIG. 2 has imaging section 10 and control analysis section 13. Imaging section 10 can observe and take an image of behavior of carrier particles 6 (described later) disposed in flow path 4 of device 2. When at least one of top substrate 21 and bottom substrate 22 of device 2 is transparent, imaging section 10 can easily take the image of the inside situation of flow path 4. Furthermore, when objective lens 11 is placed between imaging section 10 and device 2, an enlarged image of the inside situation of flow path 4 is taken. The taken image may be a moving image or a still image. Moreover, the resolution of the taken image may be increased by disposing a light source (not shown), for example.

Control analysis section 13 can control the measurement in apparatus 1 and process the image taken by imaging section 10. For example, control analysis section 13 can count the number of particles in the image.

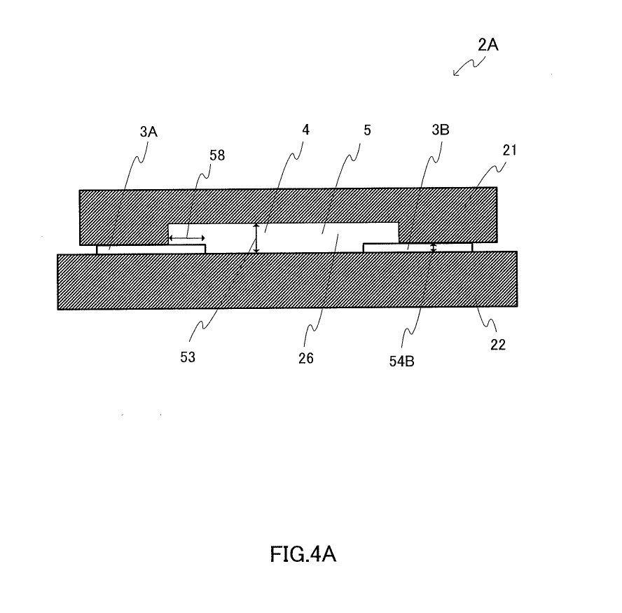

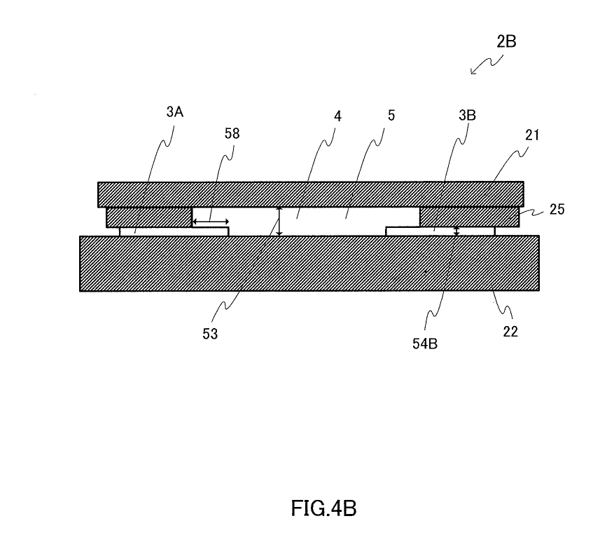

As described above, device 2 has the electrode pair, and flow path 4 serving as the reaction field is formed between the electrode pair. Device 2 includes, for example, 1) top substrate 21 with concave part 26, and bottom substrate 22 on which electrode 3A and electrode 3B are disposed, whereby electrode 3A and electrode 3B are interposed between top substrate 21 and bottom substrate 22 (see device 2A of FIG. 4A), or 2) top substrate 21, bottom substrate 22 on which electrode 3A and electrode 3B are disposed, and middle substrates 25 interposed between top substrate 21 and, electrode 3A and electrode 3B (see device 2B of FIG. 4B). FIGS. 4A and 4B are cross-sectional views of device 2, when taken along line A-A′ of FIG. 3.

As shown in FIG. 4A, flow path 4 of device 2A is formed by a space of concave part 26 of top substrate 21, and a space between electrode 3A and electrode 3B. On the other hand, as shown in FIG. 4B, flow path 4 of device 2B is formed by a space between middle substrates 25, and a space between electrode 3A and electrode 3B. In device 2B, middle substrate 25 forms one inner surface of flow path 4. In either case, flow path 4 of device 2 has a top surface formed by top substrate 21, and a bottom surface formed by bottom substrate 22. Electrode 3A and electrode 3B are exposed to the inside of flow path 4.

In device 2B, the thickness of middle substrate 25 is substantially equal to the depth of flow path 4, and therefore, it is not necessary to form concave part 26 in top substrate 21, and top substrate 21 can be formed in a flat panel shape. Therefore, device 2B is preferable from the viewpoint of production. In addition, if both sides of middle substrate 25 is adhesive, it is possible to easily attach middle substrate 25 to top substrate 21 or bottom substrate 22, so that the fabrication efficiency improves.

It is preferable that the film thickness 54B of electrodes 3A and 3B of device 2 is adjusted to 1 nm to 10 μm. In particular, when the electrodes are formed using sputtering, it is preferable that the film thickness 54B of electrodes 3A and 3B is adjusted to 50 to 500 nm, preferably 100 nm, from the viewpoint of production time. The width 56 of electrodes 3A and 3B is arbitrary, but it is usually about 0.5 mm to about 5 mm. In addition, electrodes 3A and 3B have terminals 9 that realize interconnection or electrical contact so as to communicate with voltage applying section 12.

Distance 55 between electrodes 3A and 3B is adjusted to 20 to 1,000 μm. When distance 55 is less than 20 μm, it is not possible to ensure a sufficient area to form pearl chains with particles (i.e. to connect particles in rows) with diameter of several μm. Also, when distance 55 is more than 1,000 μm, it is not preferable because an effective electric field intensity may not be acquired, or a high voltage of 50 V or more needs to be applied to obtain an effective electric field intensity. Distance 55 is preferably 100 to 500 μm, and more preferably, about 500 μm.

A material of electrodes 3A and 3B is conductive, and has characteristics of not dissolved or peeled off in a solution by application of an alternating voltage. Examples of the electrode material include gold, silver, platinum, copper, aluminum, chromium, nickel, tungsten, or an alloy thereof. When gold is used as the electrode material, a film may be formed firmly on bottom substrate 22 by a sputter technique, and the film stability is preferably increased with respect to application of an alternating voltage.

Top substrate 21 and bottom substrate 22 of device 2 may have any dimensions as long as flow path 4 and electrodes 3A and 3B can be disposed.

Top substrate 21 and bottom substrate 22 of device 2 are formed of insulating materials or semiconductor materials. Examples of the insulating materials include organic materials or inorganic insulating materials such as glass. At least one of top substrate 21 and bottom substrate 22 of device 2 is required to be transparent, and therefore, both top substrate 21 and bottom substrate 22 cannot be formed of semiconductor materials, but either of them may be formed of semiconductor materials.

Examples of the organic materials include polyethylene, polypropylene, polyisobutylene, polyethylene terephthalate (PET), unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate (PC), polyamide, phenolic resin, urea resin, epoxy resin, melamine resin, styrene-acrylonitrile copolymer, acrylonitrile-butadiene-styrene copolymer, silicone resin, polyphenylene oxide, and polysulphone.

Examples of the inorganic insulation materials include alumina, sapphire, forsterite, silicon carbide, silicon oxide, and silicon nitride. Examples of the glass include alkali glass, alkali soda glass, borosilicate glass, and silica glass. Examples of the semiconductor materials include single-crystal silicon, amorphous silicon, silicon carbide, silicon oxide, and silicon nitride.

In general, glass is excellent in heat conductivity, and therefore is suitable for a material of device 2. This is because glass can suppress increase in temperature of the reaction system by rapidly radiating the heat generated by the electric field, which is applied to form pearl chains of particles. However, according to the present invention, it is difficult to increase the temperature of the reaction system even though an electric field is applied to form pearl chains of particles, so that device 2 can be formed of a material that conducts heat lower than the glass. Therefore, top substrate 21 and bottom substrate 22 of substrate 2 can be formed of organic materials, preferably PET resin. Although the heat conduction of PET resin is much poorer than the glass, PET resin can be used as a material of the device in the apparatus according to the present invention. By forming device 2 from an organic material such as PET resin, it is possible to reduce the production cost of device 2 and improve the production processes, so that production efficiency remarkably increases.

In addition, it is preferable that a portion of at least one of top substrate 21 and bottom substrate 22 of device 2 is transparent, in order to observe the inside situation of flow path 4. In particular, if the depth is properly defined, the behavior of carrier particles 6 (described later) in flow path 4 may be recognized without defocusing the behavior. For example, by detecting agglutination of carrier particles 6 generated by an antigen-antibody reaction, it is possible to detect an antigen or an antibody and measure its quantity.

In flow path 4 serving as the reaction field of device 2, carrier particles 6 with diameter of several μm are provided. When carrier particles 6 are arranged in one layer to reduce their overlaps, a particle image taken from above of flow path 4 is neither overlapped nor defocused. Therefore, it is preferable that the depth 53 of flow path 4 is 5 μm or more and 10 μm or less.

Although the width and length of flow path 4 are arbitrarily set, the volume of flow path 4 is more than a necessary quantity of biological sample solution 5 to be injected. The width of flow path 4 is typically 0.75 mm, preferably 0.2 mm or more and 1 mm or less, and usually 0.1 mm or more and 10 mm or less. The length of flow path 4 is about 5 mm.

In flow path 4, electrode 3A and electrode 3B are partially exposed. The exposed width of the electrode is more than 0 mm and 3 mm or less, approximately between 0.1 mm and 0.2 mm.

The inner surface of flow path 4 may be hydrophilic or hydrophobic. Also, all or a portion of the surface may be hydrophilic or hydrophobic. Furthermore, the surface treatment may be treated to suppress adsorption of particles.

Top substrate 21 or bottom substrate 22 may be provided with an inlet (not shown) through which liquid 5 is injected into flow path 4, or an outlet (not shown) through which a gas or the like in flow path 4 exit. Positions and sizes of the inlet and the outlet are not particularly limited as long as the inlet and the outlet are connected to flow path 4.

Method for Producing the Device

The method for producing device 2 is not particularly limited. For example, device 2 may be produced by the following processes.

1) Process of forming electrode 3A and electrode 3B on one surface of bottom substrate 22
2) Process of forming concave part 26 in top substrate 21 according to the shape of flow path 4, or if necessary, the inlet for injection of liquid 5, and an air hole.
3) Process of attaching top substrate 21 and bottom substrate 22 to each other.

In process 1), electrodes 3A and 3B are formed on the bottom substrate by using techniques known to a person skilled in the art, such as thin film deposition technique represented by sputtering or vapor deposition, or printing technique. The sputtering is preferable because electrodes 3A and 3B may be easily formed thinly, and, when top substrate 21 and bottom substrate 22 are attached in process 3), it is difficult to form a gap due to step difference of the electrodes between top substrate 21 and bottom substrate 22.

In process 2), concave part 26, the inlet or the air hole are formed using techniques known to a person skilled in the art. For example, a cutting technique, a removal technique using laser, or an imprint technique, or a photolithography may be used.

In process 3), top substrate 21 and bottom substrate 22 are attached to each other using techniques known to a person skilled in the art. Through these processes, device 2 is made and used.

Device 2 can be produced in a manner similar to device 2, except that concave part 26 is not formed in process 2), and that top substrate 21 and bottom substrate 22 are attached to each other via middle substrate 25 in process 3).

Carrier particles 6 are provided in flow path 4 serving as the reaction field of device 2. Carrier particles 6 may be disposed in flow path 4 in advance before the sample solution is injected, or may be supplied to flow path 4 together with the sample solution. The alternating electric field applied to the reaction field forms pearl chains of particles 6.

Examples of carrier particles 6 according to the present invention include latex particles, bentonite, kaolin, gold colloid, red blood cell, gelatin, and liposome. Latex particles are preferable. Latex particles generally used in an agglutination reaction may be used. For example, polystyrene-based latex, polyvinyl toluene-based latex, or polymethacrylate-based latex may be used. Functional group monomer (monomers having —COOH, —OH, —NH₂, or —SO₃) may be copolymerized and introduced in the latex particles.

In the case of the latex particles, for example, an average grain size of carrier particles 6 may be preferably 0.5 to 10 μm. Carrier particles having an average grain size of less than 0.5 μm do not give a sufficient dielectrophoretic force and their pearl chaining is difficult. Also, in the case of carrier particles having an average grain size of more than 10 μm, it is difficult to disperse the pearl-chained carrier particles even after the voltage application is stopped. In the case of the latex particles, for example, the average grain size of carrier particles 6 is more preferably 1 to 5 μm, most preferably 2 to 3 μm.

As the concentration of carrier particles 6 of the reaction system (which includes carrier particles 6 and sample solution 5) in the reaction field is higher, the pearl chains are formed more easily and the agglutination reaction is accelerated more easily. On the other hand, as the concentration of carrier particles 6 is higher, the agglutination rate of carrier particles 6 tends to increase when carrier particles are re-dispersed in the case where a biologically specifically reactive substance is not present. Further, when the concentration of carrier particles 6 is excessively high, carrier particles 6 are easily overlapped in the reaction system, and it is not preferable for measurement of components to analyze the image of aggregated carrier particles 6. From these viewpoints, in the case of the latex particles, for example, the concentration of carrier particles 6 in the reaction system is preferably 0.01 to 1 wt %, more preferably 0.1 to 0.5 wt %, and further preferably about 0.4 wt %.

In a preferred embodiment of the present invention, a biologically specifically reactive substance to be measured or detected is an antigen and/or an antibody. In a further preferred aspect of the present invention, an antigen is used as the biologically specifically reactive substance to be measured or detected, and latex particles that sensitize an antibody to the antigen are carrier particles 6. For example, in a conventionally known method, the latex particles sensitize the antibody by adsorbing or binding the antibody into the latex particles.

Voltage applying section 12 applies two or more alternating voltages having different amplitudes. For example, voltage applying section 12 applies a first alternating voltage to form pearl chains of carrier particles 6 disposed in the reaction field (described later), and a second alternating voltage having a smaller amplitude than that of the first alternating voltage. The first alternating voltage causes a first alternating electric field to be applied to the reaction field, and the second alternating voltage causes a second alternating electric field to be applied to the reaction field.

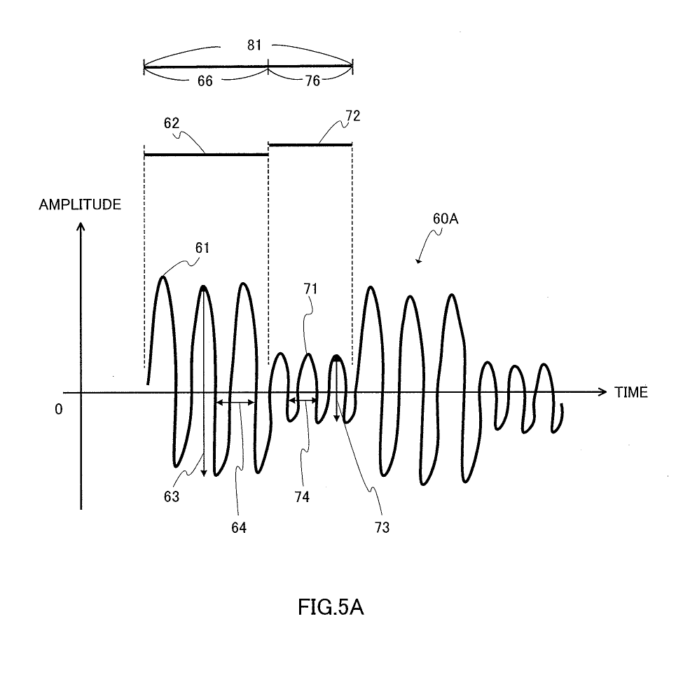

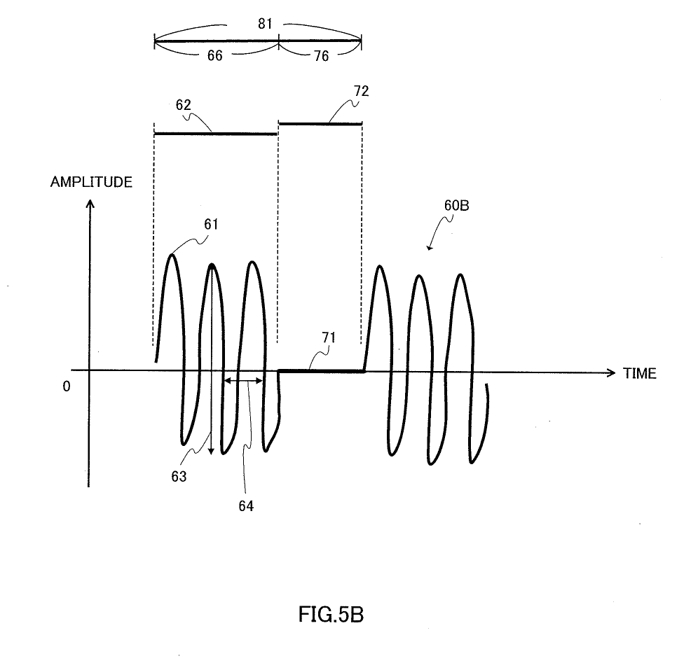

FIGS. 5A and 5B show examples of the waveforms of the alternating voltages applied by voltage applying section 12 (alternating voltage waveforms 60A and 60B). The alternating voltage waveforms 60A and 60B include first alternating voltage waveform group 62 having first amplitude value 63, and second alternating voltage waveform group 72 having second amplitude value 73, and first alternating waveform group 62 and second alternating voltage waveform group 72 are repeated. That is, total period 81 of first group period 66 and second group period 76 is repeated.

In first alternating voltage waveform group 62, first alternating voltage waveform 61 is repeated in a waveform unit during first group period 66. That is, first group period 66 is a result of multiplication between first period 64 of first alternating voltage waveform 61 and number of times voltage is applied 68.

It is preferable that first amplitude value 63 is adjusted so that the intensity of the alternating electric field applied to the reaction field is 20 V/mm or more and 100 V/mm or less. That is, when the distance between electrode 3A and electrode 3B of device 2 is 500 μm, it is preferable that first amplitude value 63 is 10 Vp-p or more and 50 Vp-p or less. Here, first amplitude value 63 refers to a peak-to-peak amplitude value.

First period 64 is preferably about 100 kHz, and is adjusted so that the frequency (first frequency) of first alternating voltage waveform 61 is 1 kHz or more and 100 MHz or less.

First group period 66 is selected from 30 nanoseconds or more and 5 seconds or less. At less than 30 nanoseconds, an effective dielectrophoretic force is not transferred to particles 6. At more than 5 seconds, the temperature increase in the reaction system is remarkable. When the frequency (first frequency) of first alternating voltage waveform 61 is 100 kHz, it is more preferable that first group period 66 is 30 μs or more and 100 ms or less. Therefore, it is preferable that the frequency of first alternating voltage waveform group 62 is 0.2 Hz or more and 1 MHz or less, and the number of times voltage is applied 68 is 3 or more and 5×10⁶ or less.

Meanwhile, in second alternating voltage waveform group 72, second alternating voltage waveform 71 is repeated in one waveform unit during second group period 76. Second group period 76 is a result of multiplication between second period 74 of second alternating voltage waveform 71 and number of times voltage is applied 78. In FIG. 5B, the second amplitude value of second alternating voltage waveform group 72 is zero.

One characteristic of second amplitude value 73 is less than first amplitude value 63. It is preferable that second amplitude value 73 is adjusted so that the electric field intensity is equal to or more than 0 V/mm and less than 20 V/mm. Therefore, when the distance between electrode 3A and electrode 3B is 500 μm, it is preferable that second amplitude value 73 is equal to or more than 0 V and less than 10 V. At 10 V or more, a sufficient dielectrophoretic force is provided to carrier particles 6 to form pearl chains, but heat is generated in the reaction system. In this case, the temperature of the reaction system increases, and therefore, a desired component measurement may not be achieved. Second amplitude value 73 is preferably 0 V at which a least amount of heat is generated (see FIG. 5B).

Second period 74 and the frequency (second frequency) of second alternating voltage waveform 71 are arbitrary.

As described above, in alternating voltage waveform 60 (60A in FIG. 5A, 60B in FIG. 5B) the combination of first alternating voltage waveform group 62 and second alternating voltage waveform group 72 is repeated at every total period 81 of group period 66 and group period 76. In this specification, the ratio between group period 66 of first alternating voltage waveform group 61 and total period 81 is referred to as “application ratio.” It is preferable that the application ratio is adjusted 0.75 or more and 0.95 or less. If the application ratio is less than 0.75, the time period for applying the second alternating voltage that does not provide a sufficient dielectrophoretic force to carrier particles 6 becomes longer. Therefore, a time period a voltage that is necessary to form pearl chains of carrier particles 6 is applied, becomes longer. On the other hand, if the application ratio is more than 0.95, the time period for applying the first alternating voltage that provides a sufficient dielectrophoretic force to carrier particles 6 becomes too long, and heat generated in the reaction system by the pearl chaining cannot be sufficiently discharged. For this reason, the temperature of the reaction system increases, and a desired component may not be measured.

First alternating voltage waveform 61 and second alternating voltage waveform 71 are arbitrary, and may be appropriately selected from a square wave, a sine wave, a cosine wave, a triangular wave and the like. It is preferable that first alternating voltage waveform 61 is a square wave.

### 2. Detecting or Measuring Method According to the Present Invention

A detecting or determining method according to the present invention (also referred to as “the method of the present invention”) includes a step of applying an alternating electric field to a reaction system containing carrier particles and a sample solution. The sample solution contains a biologically specifically reactive substance that makes a biologically specific agglutination reaction with the carrier particles. Pearl chains of the carrier particles are formed by applying the alternating electric field to the reaction system. The pearl chaining accelerates the biologically specific agglutination reaction of the biologically specifically reactive substance on the carrier particles.

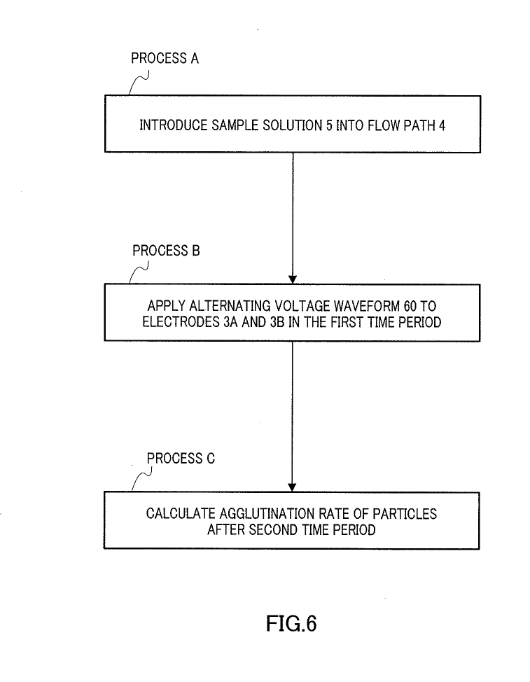

Specifically, the method of the present invention may be carried out using apparatus 1 including above-described device 2. Hereinafter, a method using pearl chaining of the carrier particles and an immune reaction will be summarized with reference to FIG. 6. This method is based on a principle of detecting the amount of antigen specifically bound to an antibody by using an agglutination rate of carrier particles 6 adsorbing the antibody.

First, sample solution 5 containing a sample to be tested is introduced into flow path 4 of device 2 (process A). Sample solution 5 filling flow path 4 at process A contacts electrodes 3A and 3B. In flow path 4, carrier particles 6 are dispersed within the solution at a predetermined concentration. In order to obtain an appropriate dispersion state, sample solution 5 containing dispersed carrier particles 6 may be introduced into flow path 4, or carrier particles 6 may be supported in flow path 4 of device 2 before sample solution 5 is introduced.

Next, voltage applying section 12 applies the voltage with alternating voltage waveform 60 to electrode 3A and electrode 3B in the first time period (process B). At process B, carrier particles 6 receive a dielectrophoresis in flow path 4 and show a chain-shaped behavior along a direction of the electric field (pearl chaining).

Then, voltage applying section 12 stops applying the voltage with alternating voltage waveform 60, and the agglutination rate of carrier particles 6 after a second time period, is calculated (process C). The agglutination rate is calculated using analysis section 13 that processes the aspect of carrier particles 6 obtained by imaging section 10. For example, in the image, the agglutination rate is calculated by the ratio between the area of the carrier particles aggregated with two or more and the area of all free particles. The agglutination rate increases according to the concentration of the sample targets contained in sample solution 5, which specifically reacts with the antibody bound to carrier particles 6, so that it is possible to detect or measure the concentration of the sample targets contained in the sample solution.

In process B, it is preferable that, in alternating voltage waveform 60 of the voltage applied by voltage applying section 12, first alternating voltage group 62 (which has first amplitude value 63) that provides a dielectrophoretic force to carrier particles 6 for alignment in a pearl chain shape, and second alternating voltage group 72 having second amplitude value 73 that is less than first amplitude value 63 are alternately repeated. By interposing second alternating voltage group 72, instead of continuously disposing first alternating voltage group 62, it is possible to release heat generated in the reaction system effectively to the outside. Further, by appropriately adjusting the application ratio (i.e. the ratio between the time period of first alternating voltage group 62 and the total of the time period of first alternating voltage group 62 and the time period of second alternating voltage group 72), it is possible to provide a dielectrophoretic force equivalent to that in the case where first alternating voltage group 62 continues, to carrier particles 6. For this reason, it is possible not to vaporize a solution even when the solution is very conductive, and possible to form pearl chains of carrier particles 6, thereby applying the carrier particles to the component measurement.

Therefore, it is possible to use the blood sample or the blood plasma sample which is very conductive and which is an example of the biological sample, as the sample solution without diluting the blood sample or the blood plasma sample in advance (preparatory process). Accordingly, apparatus 1 of the present invention need not include a previous diluting means and can have a simple structure. Also, the operator is not requested to carry out preparatory processing, so that it is possible to conduct measurement simply without errors.

Furthermore, by appropriately adjusting the amplitude values and the time periods of the first alternating voltage and the second alternating voltage, it is possible to form pearl chains of particles even in the highly conductive solution without greatly decreasing the time it takes to complete the pearl chaining. Therefore, the present invention provide an advantage of measuring the biologically specifically reactive substance rapidly.

Moreover, voltage applying section 12 applies the second alternating voltage, so that device 2 configuring the reaction system can be formed of a resin with much poorer heat conduction than glass, as well as glass with high heat conduction. Accordingly, device 2 can be produced easily at low cost and is produced easily, so that production efficiency remarkably improves.

It is preferable that electric field applying section 12 maintains the temperature in flow path 4 at 45° C. or less, 60 seconds after applying alternating voltage 60. If the temperature in flow path 4 is above 45° C., proteins contained in the sample are denatured by heat generated in the reaction system, to be fixed in the reaction system, or the proteins themselves, which are subject to measurement, are denatured, thereby causing a harmful effect of not exhibiting specific reaction such as an antigen-antibody reaction.

It is preferable that the effective value of the alternating current applied between electrode 3A and electrode 3B by electric field applying section 12 is 0 mA or more and 96 mA or less. In particular, if the effective value of the current is less than 0.7 mA, it is preferable because heat is not generated and the measurement may be conducted in a typical manner. On the other hand, if the effective value of the current is more than 96 mA, it is not preferable because heat is considerably generated. In particular, the temperature in flow path 4 in device 2 where flow path 4 is formed of a poorly heat conductive material (for example, a resin such as PET) easily increases by the voltage application. For example, there are cases where the temperature in flow path 4 becomes equal to or more than 45° C., 60 seconds after the voltage application is started (refers to Examples).

To reduce the alternating current applied to electrodes 3A and 3B at 96 mA or less, the conductivity of the solution, the shape (depth, length) of the flow path and the width of the electrodes are also important. This is because the alternating current value is determined by the applied alternating current and the resistance exhibited by the solution. The resistance exhibited by the solution is inversely proportional to the depth and length of the flow path with respect to the conductivity of the solution, and proportional to the width of the electrode. From these relationships, when the depth of the flow path is 10 μm, the length of the flow path is 4 mm, and the width of the electrode is 0.5 mm by way of example, it is preferable that the conductivity of solution 5 of the present invention is 0.1 mS/cm or more and 35 mS/cm or less.

The inside of flow path 4 is filled with sample solution 5. Sample solution 5 can be an electrolyte solution containing a predetermined concentration of salt, or a blood or blood plasma sample that is a biological sample. If sample solution 5 contains salt, the biologically specific reaction represented by the antigen-antibody reaction is stabilized. On the other hand, if sample solution 5 excessively contains salt, the conductivity of sample solution 5 increases and heat energy also increases due to voltage application. When the conductivity is less than 0.1 mS/cm, heat is not generated and the measurement can be conducted in a typical manner. However, when the conductive is more than 35 mS/cm, it is not preferable because the possibility of heat generation remarkably increases.

In the above example of the flow path, the electrolyte solution with the salt concentration up to 200 mM may be used as sample solution 5. As an example of the biological sample, the blood plasma sample solution contains a salt with the same concentration as the normal saline solution (120 mM in concentration conversion). The conductivity is about 15 mS/cm, though there are differences between individuals. Therefore, the method of the present invention may detect or measure the components of the blood plasma sample solution.

### EXAMPLES

Hereinafter, the present invention will be described more specifically with reference to examples and comparative examples. These examples do not limit the present invention.

[Preparation of Device 2-1]

Device 2-1 used in the following examples or comparative examples was a device having a middle substrate as shown in FIG. 4B. First, bottom substrate 22 was made. As a material of bottom substrate 22, a polyethylene terephthalate (PET) substrate (150 mm square, 0.1 mm in thickness) (COSMOSHINE A4300, manufactured by TOYOBO Co., Ltd.) treated clean was prepared. By sputtering through a stencil mask (made of stainless steel) penetrating along patterns of the electrode pair, gold was deposited on the PET substrate to 1,000 angstrom electrode 3A, electrode 3B, and terminals 9. The electrode width of electrode 3A and electrode 3B was 1 mm, the distance between the electrode pair was 0.5 mm, and the length of electrodes 3A and 3B was 5 mm. In this way, a pair of rectangular electrodes was made.

Then, top substrate 21 was made. A similar PET substrate was prepared as top substrate 21. In top substrate 21, through-holes with diameter of 1 mm were formed as an inlet and an outlet.

Next, middle substrate 25 was made. Middle substrate 25 was a double-coated adhesive sheet (Nitto Denko Corporation No. 5601) with a total thickness of 10 μm, and an acrylic adhesive was coated on both sides of a polyester substrate. The through-holes were formed in a shape corresponding to the shape (rectangular shape) of flow path 4. The width of flow path 4 was 0.75 mm, and the length of flow path 4 was 10 mm.

Device 2 was made by attaching bottom substrate 22 and top substrate 21, with middle substrate 25 being interposed therebetween.

[Preparation of Device 2-2]

Device 2-2 used in the following examples or comparative examples was a substrate with a two-sheet structure that includes top substrate 21 having concave part 26, and bottom substrate 22.

Bottom substrate 22 was made with borosilicate glass (SCHOTT AG D-263) (0.1 mm in sheet thickness). Electrode 3A and electrode 3B were formed by sputtering gold on bottom substrate 22. For the purpose of improving adhesion between glass and gold, chromium was deposited to 50 angstrom, and then gold was deposited to 950 angstrom. The width and length of the electrodes, and the distance between the electrode pair were the same as those of device 2-1.

Top substrate 21 was made with borosilicate glass (SCHOTT AG D-263) (0.1 mm in sheet thickness). A concave part was formed by photolithography and wet etching. The depth, width and length, of the flow path were the same as those of device 2-1.

Device 2-2 was made by attaching top substrate 21 and bottom substrate 22 using UV curable adhesive.

[Preparation of Device 2-3]

Device 2-3 used in the following examples or comparative examples was a device with a middle substrate as shown in FIG. 4B. Device 2-3 was made in a manner similar to device 2-1, except that top substrate 21 and bottom substrate 22 were made using glass plates having the same component as in Comparative example 2.

In the following Table 1, the structures of substrates 2-1 to 2-3 are summarized. Device 2-1 is device 2A in which all the side faces of flow path 4 were formed of PET. Specifically, top substrate 21, middle substrate 25, and bottom substrate 22 were formed of PET, polyester, and PET, respectively. In the case of device 2-2, all the sides of flow path 4 were formed of glass. In the case of device 2-3, top substrate 21, middle substrate 25, and bottom substrate 22 were formed of glass, polyester, and glass, respectively. Numbers within parentheses in Table 1 represent heat conductivities (λW/(mK)) of components.

TABLE 1

| | | | DEVICE 2-1 | DEVICE 2-2 | DEVICE 2-3 |
|---|---|---|---|---|---|
| MATERIAL OF | TOP SURFACE | TOP | PET | GLASS | GLASS |
| DEVICE 2 | OF FLOW PATH | SUBSTRATE 21 | (0.2) | (0.81) | (0.81) |
| | SIDE SURFACE | | — | GLASS | — |
| | OF FLOW PATH | | | (0.81) | |
| | | MIDDLE | POLYESTER | — | POLYESTER |
| | | SUBSTRATE 25 | (0.2) | | (0.2) |
| | BOTTOM SURFACE | BOTTOM | PET | GLASS | GLASS |
| | OF FLOW PATH | SUBSTRATE 22 | (0.2) | (0.81) | (0.81) |

### Example 1

In Example 1, the alternating voltage of alternating voltage waveform 60B generating first alternating voltage waveform 61 intermittently, as shown in FIG. 5B, was applied to the electrode pair of device 2-1. The temperature in flow path 4 of device 2-1 when the alternating voltage of alternating voltage waveform 60B was applied was measured, and the behavior of carrier particles 6 provided inside flow path 4 was observed.

Alternating voltage waveform 60B applied in Example 1 was set as follows. Regarding first alternating voltage waveform 61A, the first frequency was set to 100 kHz, and the first amplitude value was set to 20 Vp-p. Regarding the second alternating voltage waveform, the second amplitude value was set to 0 Vp-p (i.e. the second frequency 75A was 0 Hz).

In addition, the application ratio (ratio between the application time period of the first alternating electric field and the total of the application time period of the alternating electric field) was set in four types of Examples 1a to 1d. In each of Examples 1a to 1d, a total period 81 of the alternating voltage was 100 milliseconds. In Example 1a, the application ratio was set to 0.9. One period of the first alternating voltage waveform group was set to 90 milliseconds, and one period of the second alternating voltage waveform group was set to 10 milliseconds. In Example 1b, the application ratio was set to 0.8. One period of the first alternating voltage waveform group was set to 80 milliseconds, and one period of the second alternating voltage waveform group was set to 20 milliseconds. In Example 1c, the application ratio was set to 0.75. One period of the first alternating voltage waveform group was set to 75 milliseconds, and one period of the second alternating voltage waveform group was set to 25 milliseconds. In Example 1d, the application ratio was set to 0.5. One period of the first alternating voltage waveform group was set to 50 milliseconds, and one period of the second alternating voltage waveform group was set to 50 milliseconds.

### Comparative Example 1

In Comparative example 1, the alternating voltage of the alternating voltage waveform generated by continuously oscillating only the first alternating voltage waveform of Example 1, was applied to the electrode pair of device 2-1. Like Example 1, the temperature in flow path 4 was measured, and the behavior of carrier particles 6 provided inside flow path 4 was observed.

### Comparative Example 2

In Comparative example 2, the alternating voltage of the alternating voltage waveform generated by continuously oscillating only the first alternating voltage waveform of Example 1, was applied to the electrode pair of device 2-2 (device using a glass substrate). Like Example 1, the temperature in flow path 4 was measured, and the behavior of carrier particles 6 provided inside flow path 4 was observed.

The alternating voltages applied in Comparative examples and 2 were set as follows. Regarding the first alternating voltage waveform, as in Example 1, the first alternating voltage waveform was continuously oscillated by setting the first frequency to 100 kHz and the first amplitude value to 20 Vp-p. Therefore, the application ratio was 1. The alternating voltage waveforms applied in Example 1 and Comparative example 1 were all sine waves, and the first time period the alternating voltage is applied was set to 60 seconds.

Conditions of Example 1 and Comparative examples 1 and 2 are summarized in Table 2. Numbers within parentheses of the material of device 2 in Table 1 represent heat conductivities (W/(mK)) of components.

TABLE 2

| | | EXAMPLE 1 | | | | COMPARATIVE | COMPARATIVE |
|---|---|---|---|---|---|---|---|
| | | a | b | c | d | EXAMPLE 1 | EXAMPLE 2 |
| ALTERNATING | OSCILLATION TYPE | INTERMITTENT | | | | CONTINUOUS | CONTINUOUS |
| VOLTAGE | APPLICATION RATIO | 0.9 | 0.8 | 0.75 | 0.5 | 1 | 1 |

| | | |
|---|---|---|
| WAVEFORM | FIRST VOLTAGE | 20 |
| | AMPLITUDE VALUE(Vp-p) | |
| | FIRST FREQUENCY (kHz) | 100 |

| | | | |
|---|---|---|---|
| DEVICE 2 | DEVICE 2-1 | DEVICE 2-1 | DEVICE 2-2 |

[Experiment for Observing Behavior of Carrier Particles and Measuring Temperature According to Voltage Application]

An experiment for measuring the temperature in flow path 4 and observing the behavior of carrier particles 6 when the voltage was applied to the electrode pairs of device 2-1 and device 2-2, was conducted. Latex beads (Bangs Laboratories, Inc) adsorbing anti-CRP polyclonal antibodies and having an average diameter of 2 μm were used as carrier particles 6. A solution containing 1×10⁻² M of antigens specifically bound to the antibodies adsorbed into carrier particles 6 was used as sample solution 5. Kinds of the antibodies are not particularly limited, and, for example, anti-myoglobin antibody, anti-CRP antibody, or anti-hemoglobin A1c antibody may be used. The composition of solution 5 was 150 mM NaCl, 20 mM glycine with pH 8.6, and 0.1% BSA. Solution 5 was diluted with a buffer solution so that the final concentration of carrier particles 6 was 0.4%. The concentration of antigen, which carries out the specific reaction, and which is contained in sample solution 5, is extremely low. Therefore, even if the agglutination reaction of carrier particles 6 is accelerated by pearl chaining, it is expected that the agglutination rate of carrier particles 6 becomes low by redispersion.

First, carrier particle solution of 1 μl, was supplied from the inlet. Voltage applying section 12 (waveform generator: Hewlett-Packard Company 33120A) was used to apply the alternating voltages of the respective alternating voltage waveforms to the electrode pair of device 2 for 60 seconds. Where necessary, the voltages were amplified by connecting, for example, a power amplifier (NF Electronics Instruments 4055 High Speed Power Amplifier) next to the waveform generator.

The temperature in flow path 4 and the agglutination rate of carrier particles 6 were measured immediately after the voltage application was stopped after continuous voltage application for 60 seconds, and 60 seconds after the stop of the voltage application.

The temperature in flow path 4 was measured in a contactless manner by arranging a thermoviewer (NEC SAN-EI TH9100) above. Meanwhile, the behavior of carrier particles 6 was observed with transmitted light by using an inverted microscope manufactured by Olympus.

The depth of flow path 4 was defined as 10 μm, and therefore carrier particles 6 do not overlap with each other in two layers, and the contour of the carrier particles was clearly observed without deviation from the focus of the microscope. When carrier particles 6 received a dielectrophoresis in flow path 4, the behavior (pearl chaining) in which carrier particles 6 were aligned in a chain shape in flow path 4 was observed.

The agglutination rate was calculated from the image taken by imaging section 10, based on the following equation. The average of three screens taken by imaging section 10 is described as the agglutination rate in Table 3.

Agglutination rate (%)=(area of particles agglutinated with two or more)/(area of total particles)×100

Regarding Example 1 and Comparative examples 1 and 2, the temperature in flow path 4 and the agglutination rate are shown in Table 3. Numbers within parentheses of the internal temperature and the agglutination rate represent the internal temperature and the agglutination rate before the voltage is applied.

TABLE 3

| | | EXAMPLE 1 | | | | COMPARATIVE | COMPARATIVE |
|---|---|---|---|---|---|---|---|
| | | a | b | c | d | EXAMPLE 1 | EXAMPLE 2 |
| IMMEDIATELY | INTERNAL | 36.0 ± 0 | 33.0 ± 1.2 | 32.7 ± 0.8 | 30.8 ± 0.3 | 54.3 ± 2.8 | 30.5 ± 0.5 |
| AFTER VOLTAGE | TEMPERATURE (° C.) | (+8.5) | (+5.5) | (+5.2) | (+3.3) | (+27.0) | (+3.0) |
| APPLICATION | AGGLUTINATION | 68.8 ± 3.2 | 61.1 ± 0.7 | 47.6 ± 1.8 | 35.0 ± 2.1 | 70.9 ± 2.9 | 71.4 ± 3.5 |
| FOR 60 | RATE (%) | (+48.2) | (+42.4) | (+25.8) | (+14.9) | | (+49.9) |
| SECONDS | | | | | | | |
| 60 SECONDS | INTERNAL | 27.6 ± 0.2 | 27.5 ± 0.1 | 27.6 ± 0.3 | 27.4 ± 0.1 | 27.3 ± 0.5 | 27.7 ± 0.5 |
| AFTER | TEMPERATURE (° C.) | (+0.1) | (+0) | (+0.1) | (−0.1) | (+0) | (+0.2) |
| COMPLETION OF | AGGLUTINATION | 20.8 ± 2.0 | 20.2 ± 1.8 | 22.0 ± 2.3 | 20.5 ± 2.0 | MEASUREMENT | 21.8 ± 1.5 |
| VOLTAGE | RATE (%) | (+0.2) | (+0.5) | (−0.2) | (+0.4) | IMPOSSIBLE | (+0.3) |
| APPLICATION | | | | | | | |

Comparing Example 1 with Comparative example 1, there was a difference in the temperature in flow path 4 immediately after the voltage was applied. In Comparative example 1, the internal temperature was increased up to 54° C. with the voltage application. Although particles 6 were arranged within flow path in a pearl chain shapes, vaporization of the solution or generation of bumping-shaped bubbles was observed. 20 seconds after the voltage was applied, particles 6 were fixed in flow path 4 in the pearl chain shapes. Therefore, even when the voltage application was stopped, pearl-chain shaped carrier particles 6 were not moved, and the agglutination rate was still high even at 60 seconds after the stop of the voltage application. On the other hand, in Examples 1a to 1d, the increase of the internal temperature with the voltage application was remarkably suppressed as compared with Comparative example 1. Particles 6 were aligned in flow path 4 similarly in the pearl chain shape, and particles 6 were normally dispersed when the voltage application was stopped. That is, the agglutination rate became lower at 60 seconds after the stop of the voltage application.

In Comparative example 2, the internal temperature was increased less by the voltage application than in Comparative example 1. Carrier particles 6 were arranged within flow path 4 in the pearl chain shape, and particles 6 were normally dispersed when the voltage application was stopped. The agglutination rate became lower at 60 seconds after the stop of the voltage application.

As can be seen from the results, in Comparative example 1, carrier particles 6 were fixed by the increase in the temperature in flow path 4, and the measurement of the specific reaction such as the antigen-antibody reaction was impossible. However, in Example 1, carrier particles 6 were not fixed, and the specific reaction could be measured. The fixation of carrier particles 6 was observed when the internal temperature was more than 45° C. The internal temperature was able to be maintained at 45° C. or less according to the alternating voltage of the alternating voltage waveform of Example 1.

Also, the agglutination rate immediately after the voltage application is stopped varied depending on the application ratio. That is, the agglutination rates in Examples 1a and 1b immediately after the voltage application was stopped were almost equal to that in Comparative example 1. The agglutination rates in Examples 1c and 1d immediately after the voltage application was stopped were lower than that in Comparative example 1. However, it was confirmed that, when the voltage application period was extended, the agglutination rate was increased up to the same level as in Comparative example 1. It is found out from those results that, by setting the application ratio to 0.8 to 0.9, pearl chain can be sufficiently formed in a short time (about 2 minutes) and the specific reaction can be rapidly measured.

### Example 2

Using devices 2-1 to 2-3, the temperature in flow path 4 and the agglutination rate of carrier particles 6 when the application ratio of the alternating voltage and the amplitude value 63 of the first alternating voltage waveform were adjusted, were measured.

Table 4 shows the results when the application ratio of the alternating voltage was adjusted to 0.8, 0.5, and 0.3, and the amplitude value of the first alternating voltage waveform was adjusted to 20 V to 40 Vp-p in the case of device 2-1.

TABLE 4

| | | RATIO | | | | | | | |
|---|---|---|---|---|---|---|---|---|---|
| | | 0.8 | | | 0.5 | | | 0.3 | |
| | | AMPLITUDE VALUE | | | | | | | |
| DEVICE 2-1 | | 20 | 25 | 30 | 20 | 25 | 30 | 30 | 40 |
| IMMEDIATELY AFTER | INTERNAL | 33 | 36 | 70 | 33 | 34 | 35 | 32 | 36 |
| VOLTAGE APPLICATION | TEMPERATURE (° C.) | | | | | | | | |
| FOR 60 SECONDS | AGGLUTINATION | 61.1 | 78.3 | 86 | 48 | 70 | 71.7 | 38.5 | 80.9 |
| | RATE (%) | | | | | | | | |
| 60 SECONDS AFTER | INTERNAL | 27.5 | 27.3 | 27.3 | 27.6 | 27.6 | 27.5 | 27.5 | 27.6 |
| COMPLETION OF | TEMPERATURE (° C.) | | | | | | | | |
| VOLTAGE APPLICATION | AGGLUTINATION | 20.2 | 20 | MEASUREMENT | 20.2 | 20.5 | 22.0 | 18.3 | 25 |
| | RATE (%) | | | IMPOSSIBLE | | | | | |

As shown in Table 4, in device 2-1, the internal temperature became 45° C. or less, the fixation of carrier particles 6 was suppressed, and the specific reaction such as the antigen-antibody reaction could be measured, by 1) setting the first amplitude value to 25 Vp-p or less when the application ratio was 0.8, 2) setting first amplitude value 63 to 30 Vp-p or less when the application ratio was 0.5, and 3) setting the first amplitude value to 40 Vp-p or less when the application ratio was 0.3.

Also, in device 2-1, 1) the agglutination rate immediately after the voltage is applied was 78% when the application ratio was 0.8 and the first amplitude value was 25 Vp-p, 2) the agglutination rate immediately after the voltage is applied was 72% when the application ratio was 0.5 and the first amplitude value was 30 Vp-p, and 3) the agglutination rate immediately after the voltage is applied was 81% when the application ratio was 0.3 and the first amplitude value was 40 Vp-p. It is found out that the specific reaction such as the antigen-antibody reaction could be measured in a short time, for example, about 2 minutes.

Table 5 shows the results when the application ratio of the alternating voltage was adjusted to 1 and 0.8, and the amplitude value of the first alternating voltage waveform was adjusted to 20 V to 50 Vp-p in the case of device 2-2.

TABLE 5

| | | RATIO | | | | | |
|---|---|---|---|---|---|---|---|
| | | 1 | | | | | 0.8 |
| | | AMPLITUDE VALUE | | | | | |
| DEVICE 2-2 | | 20 | 25 | 30 | 40 | 50 | 50 |
| IMMEDIATELY | INTERNAL | 31 | 30 | 35 | 42 | 50 | 31 |
| AFTER VOLTAGE | TEMPERATURE (° C.) | | | | | | |
| APPLICATION FOR | AGGLUTINATION | 72 | 89.5 | 91.2 | 91.7 | 95.2 | 83.8 |
| 60 SECONDS | RATE (%) | | | | | | |
| 60 SECONDS AFTER | INTERNAL | 27.5 | 27.3 | 27.2 | 27.6 | 27.6 | 27.6 |
| COMPLETION OF | TEMPERATURE (° C.) | | | | | | |
| VOLTAGE | AGGLUTINATION | 20 | 20.4 | 21 | 20.2 | MEASUREMENT | 21.2 |
| APPLICATION | RATE (%) | | | | | IMPOSSIBLE | |

All the surfaces of flow path 4 of device 2-2 were formed of glass. Even though the alternating voltage of the continuously oscillated alternating voltage waveform was applied, the temperature increase was suppressed as compared with device 2-1. However, the internal temperature became 45° C. or more (50° C.) and carrier particles 6 were fixed and the measurement could not be conducted when first amplitude value 63 was more than 50 Vp-p. On the other hand, when the application ratio was set to 0.8, the internal temperature did not increase, and the specific reaction such as the antigen-antibody reaction was able to be measured.

Table 6 shows the results when the application ratio of the alternating voltage was adjusted to 1 and 0.8, and the amplitude value of the first alternating voltage waveform was adjusted to 20 V to 40 Vp-p in the case of device 2-3.

TABLE 6

| | | RATIO | | | | |
|---|---|---|---|---|---|---|
| | | 1 | | | | 0.8 |
| | | AMPLITUDE VALUE | | | | |
| DEVICE 2-3 | | 20 | 25 | 30 | 40 | 40 |
| IMMEDIATELY | INTERNAL | 30 | 35 | 41.5 | 48 | 32 |
| AFTER VOLTAGE | TEMPERATURE (° C.) | | | | | |
| APPLICATION | AGGLUTINATION | 69.4 | 74.3 | 74.4 | 74.1 | 78.5 |
| FOR 60 SECONDS | RATE (%) | | | | | |
| 60 SECONDS AFTER | INTERNAL | 27.5 | 27.5 | 27.6 | 27.6 | 27.4 |
| COMPLETION OF | TEMPERATURE (° C.) | | | | | |
| VOLTAGE | AGGLUTINATION | 20.6 | 22 | 22.5 | MEASUREMENT | 20.8 |
| APPLICATION | RATE (%) | | | | IMPOSSIBLE | |

In flow path 4 of device 2-3, two surfaces (top surface and bottom surface) were formed of glass. When the alternating voltage of the continuously oscillated alternating voltage waveform was applied, the temperature increased as compared with that of device 2-2. When the first amplitude value was 40 Vp-p or more, the internal temperature became 45° C. or more (48° C.), and carrier particles 6 were fixed and the measurement could not be conducted. On the other hand, when the alternating voltage of the alternating voltage waveform with the application ratio being set to 0.8, the internal temperature did not increase, and the specific reaction such as the antigen-antibody reaction was able to be measured.

It is found out from the above results that device 2 in measuring apparatus 1 may be made using a resin (PET), and a simple method using middle substrate 25 shown in FIG. 3B can be applied.

### Example 3

Using device 2-1 or device 2-2, the measurement using the pearl chaining reaction and the biologically specific reaction represented by the antigen-antibody reaction was verified. A solution prepared by dissolving C-reactive protein (hereinafter referred to as “CRP”), which is contained in blood, and which is used as marker protein of various inflammations, in the above-described buffer solution at a different concentration, was used as sample solution 5. To carrier particles 6, anti-CRP polyclonal antibody (hereinafter referred to as “anti-CRP antibody”) specifically bound with the CRP was sensitized. The antigen concentration M of sample solution 5 was 1×10̂X (where X=−5, −7, −9, −11). The concentration of carrier particles 6 was the same as in Example 1.

First, sample solution 5 containing the CRP and carrier particles 6 were mixed to react at room temperature for 90 seconds. Second, 1 μL sample solution 5 containing carrier particles 6 after the reaction was injected into flow path 4. The alternating voltage under the conditions shown in Table 7 was applied for 60 seconds. Finally, at 60 seconds after the completion of the voltage application, the agglutination rate was calculated. The application ratio of the alternating voltage was set to 0.8, and the first amplitude value was set to 25 Vp-p. The measurement results when device 2-1 and device 2-2 were used are shown in Table 7.

TABLE 7

| | |
|---|---|
| DEVICE 2-1 | DEVICE 2-2 |

| | | |
|---|---|---|
| ALTERNATING | OSCILLATION TYPE | INTERMITTENT |
| VOLTAGE WAVEFORM | APPLICATION RATIO | 0.8 |
| | FIRST VOLTAGE AMPLITUDE | 20 |
| | VALUE (Vp-p) | |
| | FIRST FREQUENCY (kHz) | 100 |

| | | | |
|---|---|---|---|
| AGGLUTINATION | ANTIGEN CONCENTRATION (M) 5 × 10⁻⁵ | 81.2 ± 2.4 | 87.2 ± 1.0 |
| RATE (%) | ANTIGEN CONCENTRATION (M) 5 × 10⁻⁷ | 66.5 ± 6.8 | 69.7 ± 2.1 |
| | ANTIGEN CONCENTRATION (M) 5 × 10⁻⁹ | 20.3 ± 4.4 | 15.9 ± 2.4 |
| | ANTIGEN CONCENTRATION (M) 5 × 10⁻¹¹ | 17.1 ± 1.4 | 16.5 ± 2.1 |

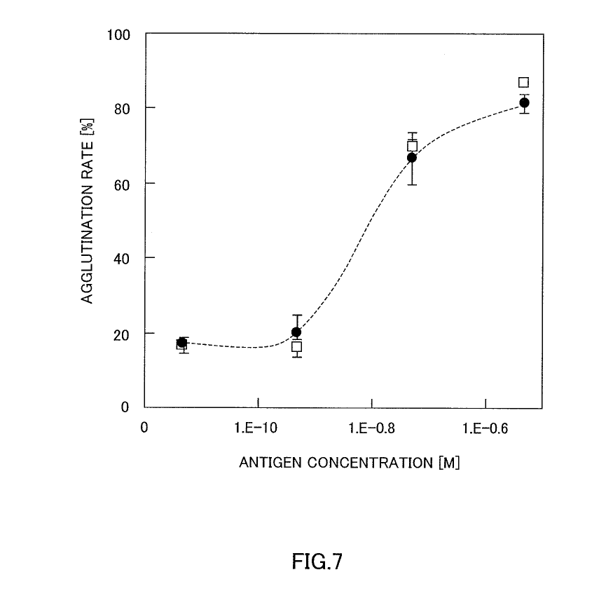

In the case of using device 2-1, similar to the case of using device 2-2, the agglutination rate varied depending on the antigen concentration. In the graph of FIG. 7, the horizontal axis represents the antigen concentration (M), the vertical axis represents the agglutination rate (%), “•” (black circle) represents a plot when device 2-1 was used, and “□” (white square) represents a plot when device 2-2 was used. From these results, it was shown that using device 2-1, the biologically specific reaction represented by the antigen-antibody reaction could be measured. In this way, even when device 2 of apparatus 1 according to the embodiment of the present invention was formed of PET (resin), it is possible to measure the biologically specific reaction represented by the antigen-antibody region.

### Example 4

In Example 4, the blood plasma sample, which is an example of a biological sample, was used as sample solution 5. Specifically, using device 2-1 with flow path 4 formed of a resin, the temperature in flow path 4 was measured, the behavior of carrier particles 6 was observed, and the biologically specific reaction was detected. The case of Example 1 where the intermittently oscillated alternating voltage waveform was applied, and the case where the continuously oscillated alternating voltage waveform was applied were compared with each other. The measurement conditions and the measurement results are summarized in Table 8.

TABLE 8

| | | DEVICE | |
|---|---|---|---|
| | | DEVICE 2-1 | DEVICE 2-1 |
| SAMPLE SOLUTION 5 | | BLOOD PLASMA | |
| | | (UNDILUTED) | |
| ALTERNATING | OSCILLATION TYPE | INTERMITTENT | CONTINUOUS |
| VOLTAGE | APPLICATION RATIO | 0.8 | 1 |

| | |
|---|---|
| FIRST VOLTAGE | 30 |
| AMPLITUDE VALUE(Vp-p) | |
| FIRST FREQUENCY (kHz) | 100 |

| | | | |
|---|---|---|---|
| IMMEDIATELY AFTER | INTERNAL TEMPERATURE (° C.) | 42 | 96 |
| VOLTAGE APPLICATION | AGGLUTINATION RATE (%) | 87.4 | MEASUREMENT |
| FOR 60 SECONDS | | | IMPOSSIBLE |
| 60 SECONDS AFTER | INTERNAL TEMPERATURE (° C.) | 27.6 | 27.3 |
| COMPLETION OF | AGGLUTINATION RATE (%) | 30.6 | MEASUREMENT |
| VOLTAGE APPLICATION | | | IMPOSSIBLE |

As shown in Table 8, it is found out that components could be measured in the undiluted blood plasma sample by applying the voltage of the alternating voltage waveform.

The disclosure of Japanese Patent Application No. 2007-274194, filed on Oct. 22, 2007, including the specification, drawings and abstract, is incorporated herein by reference in its entirety.

### INDUSTRIAL APPLICABILITY

The apparatus for detecting or measuring biologically specifically reactive substance according to the present invention is suitable for use in a device for analyzing components produced by a living organism such as biological samples and, in particular, proteins included in blood. Therefore, the present invention is applicable for use in POCT (Point of care test) biosensors that separate, purify, react and detect proteins and materials that serve as an indicator of health contained in a blood sample.

### REFERENCE NUMERALS

1 Apparatus
2 Device
3A, 3B Electrode
4 Flow path
5 Sample solution
6 Carrier particles
9 Terminal
10 Imaging section
11 Objective lens
12 Voltage applying section
13 Control analysis section
14 Connector
21 Top substrate
22 Bottom substrate
25 Middle substrate
26 Concave part
53 Depth (thickness) of flow path
54B Height (thickness) of electrode
55 Distance between electrodes
56 Width of electrode
58 Exposed width of electrode
60A, 60B Alternating voltage waveform
61 First alternating voltage waveform
62 First alternating voltage waveform group
63 First amplitude value (peak-to-peak amplitude value)
64 Period of first alternating voltage waveform
66 Group period of first alternating voltage waveform group
71 Second alternating voltage waveform
72 Second alternating voltage waveform group
73 Second amplitude value (peak-to-peak amplitude value)
74 Period of second alternating voltage waveform
76 Group period of second alternating voltage waveform group
81 Period of total alternating voltages (total of group period of first alternating voltage waveform group and group period of second alternating voltage waveform group)
101, 102 Slide glass
103, 104 Electrode

1-14. (canceled)

15. An apparatus for detecting or measuring a biologically specifically reactive substance by a biologically specific agglutination reaction on carrier particles, the apparatus comprising:
a device including a pair of electrodes and a reaction field disposed between the pair of electrodes; and
a voltage applying section that applies an alternating voltage to the pair of electrodes such that pearl chains of carrier particles disposed in the reaction field are formed,
wherein the device includes a flow path forming the reaction field;
at least one surface of the flow path is formed of a resin; and
the voltage applying section applies at least two alternating voltages having different amplitudes.

16. The apparatus according to claim 15,
wherein the voltage applying section alternately applies a first alternating voltage with a first amplitude, and a second alternating voltage with a second amplitude; and
the first alternating voltage applies a first alternating electric field having an electric field intensity of 20 V/mm or more and 100 V/mm or less is applied to the reaction field, and the second alternating voltage applies a second alternating electric field having an electric field intensity lower than the first electric field intensity is applied to the reaction field.

17. The apparatus according to claim 16,
wherein the amplitude of the second alternating voltage is 0 V.

18. The apparatus according to claim 16,
wherein a repetition period including a time period the first alternating voltage is applied and a time period the second alternating voltage is applied is 30 nanoseconds or more and 5 seconds or less.

19. The apparatus according to claim 18,
wherein the ratio between the time period the first alternating voltage is applied and the repetition period is 75% to 95%.

20. The apparatus according to claim 15, wherein the device comprises:
a top substrate forming a top surface of the flow path, a substrate shape of a top surface side being a flat panel;
a bottom substrate forming a bottom surface of the flow path, a substrate shape of the bottom surface side being a flat panel; and
a middle substrate forming a side surface of the flow path and including a penetrating region corresponding to the shape of the flow path, and
wherein at least the middle substrate is formed of a resin.

21. The apparatus according to claim 20,
wherein a surface of the middle substrate which does not form the flow path is adhesive.

22. The apparatus according to claim 15,
wherein all surfaces of the flow path are formed of a resin.

23. A method for detecting or measuring a biologically specifically reactive substance, the method comprising the steps of:
preparing a device including a pair of electrodes and a reaction field disposed between the pair of electrodes;
providing the reaction field with carrier particles and a target solution containing the biologically specifically reactive substance capable of a biologically specific agglutination reaction on the carrier particles; and
forming pearl chains of the carrier particles by applying an alternating voltage to the reaction field, wherein:
the device includes a flow path fanning the reaction field;
at least one surface of the flow path is formed of a resin; and
the reaction field is applied at least two alternating electric fields having different electric field intensities.

24. The method according to claim 23,
wherein, when the alternating electric fields are applied, an effective value of a current flowing in the target solution is 0.7 mA or more and 96 mA or less.

25. The method according to claim 23,
wherein the target solution has a conductivity of 0.1 mS/cm or more and 35 mS/cm or less.

26. The method according to claim 23,
wherein, when the alternating electric fields are applied, a temperature of the target solution is maintained at 45° C. or less.

27. The method according to claim 23,
wherein the target solution includes blood or blood plasma.
